(19) United States
(12) Patent Application Publication
(10) Pub. No.: US 2025/0381137 A1
(43) Pub. Date: Dec. 18, 2025

(54) PARABEN-FREE FEXOFENADINE FORMULATIONS

(71) Applicant: Opella Healthcare Group SAS, GENTILLY (FR)

(72) Inventors: Daniela Maldonado CAVALARI, Compiegne (FR); Sabrina BARATIERI, Sao Paulo (BR); Cleber CAMPOS, Sao Paulo (BR)

(21) Appl. No.: 18/879,185

(22) PCT Filed: Jun. 19, 2023
(86) PCT No.: PCT/EP2023/066500

§ 371 Date: Dec. 26, 2024

Related U.S. Application Data
(60) Provisional application No. 63/440,356, filed on Jan. 20, 2023.

(51) Int. Cl. A61K 9/10; A61K 31/445; A61K 47/02; A61K 47/10; A61K 47/12; A61K 47/18; A61K 47/22; A61K 47/26; A61K 47/36; A61K 47/46

(52) U.S. Cl. CPC A61K 9/10; A61K 31/445; A61K 47/02; A61K 47/10; A61K 47/12; A61K 47/183; A61K 47/186; A61K 47/22; A61K 47/26; A61K 47/36; A61K 47/46

(57) ABSTRACT

Provided is a paraben-free aqueous pharmaceutical suspension formulation comprising fexofenadine zwitterionic dihydrate Form I of formula (I) and uses thereof. In certain embodiments, the formulation comprises polypropylene glycol, edetate disodium, potassium sorbate, xanthan gum, poloxamer 407, titanium dioxide, sodium phosphate monobasic monohydrate, sodium phosphate dibasic heptahydrate, artificial raspberry cream flavor, sucrose, xylitol, and purified water. The disclosure also includes methods of making such formulations.

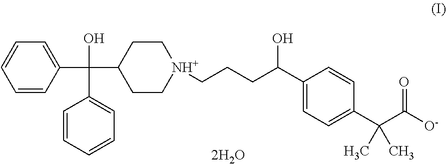 [SMILES: CC(C)(C(=O)[O-])c1ccc(C(O)CCC[NH+]2CCC(C(O)(c3ccccc3)c3ccccc3)CC2)cc1]

### CROSS-REFERENCE TO RELATED APPLICATIONS

This application claims priority to U.S. Provisional Application No. 63/440,356 (filed Jan. 20, 2023), the entire contents of which is incorporated by reference herein.

### FIELD OF THE INVENTION

This disclosure is directed to paraben-free liquid formulations of fexofenadine and uses of such formulations.

### BACKGROUND OF THE INVENTION

Fexofenadine hydrochloride refers to 4-[1-hydroxy-4-[4-(hydroxydiphenylmethyl)-1-piperidinyl]-butyl]-α,α-dimethyl benzeneacetic acid hydrochloride. It has the below structure and is available in products such as Goodsense® Aller-ease, Aller-Fex™, Wal-Fex® Allergy, Allegra®, Allegra® Allergy 12 Hour, Allegra® Allergy 24 Hour, Children's Allegra® Allergy, and Mucinex Allergy, Allegra® Allergy, and Children's Wal-Fex®. Fexofenadine generally has been known to have low solubility and low permeability

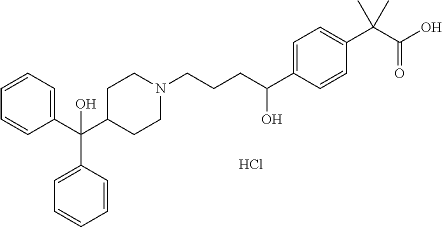 [SMILES: CC(C)(C(=O)O)c1ccc(C(O)CCCN2CCC(C(O)(c3ccccc3)c3ccccc3)CC2)cc1 ; Cl]

Fexofenadine hydrochloride has also been known to have a bitter taste that can be unpalatable. The current commercially available suspension formulation of fexofenadine (Allegra®) contains inter alia propyl paraben and butyl paraben. The propyl paraben and butyl paraben were used as a preservative system because at the time of development, they were commonly used for oral liquid dosage forms, and because of their microbial activity and stability at the drug product pH (≈6.2), and their broad spectrum of activity.

Specifically, this suspension is a white uniform suspension, typically contains 6 mg fexofenadine hydrochloride per mL and the following excipients: polypropylene glycol, edetate disodium, propylparaben, butylparaben, xanthan gum, poloxamer 407, titanium dioxide, sodium phosphate monobasic monohydrate, sodium phosphate dibasic heptahydrate, artificial raspberry cream flavor, sucrose, xylitol, and purified water. This suspension was especially designed to provide optimal stability and to mask the bitter taste associated with fexofenadine.

Parabens were used to prevent and reduce the growth of harmful bacteria and mold, increasing the shelf life of products. In recent years, formulators have begun removing parabens.

However, “[f]inding substitute ingredients to parabens, needing to be both efficient against microbiological contamination, and ensuring the safety of formulas and the success of products to consumers, is not an easy task.” Frick, R. “Formulating without parabens?Alternatives to handle with care,” Premium Beauty News (Jul. 23, 2011) (available online at www.premiumbeautynews.com/en/formulating-without-parabens, 3268 (last accessed Jan. 18, 2023). “Unfortunately, there aren't any universal solutions and work must be carried out on a case by case basis with regard to the type of formula to protect, its nature, its pH, its method of use.” Id. (quoting Jean-Pierre Arnaud, CEO of Lucas Meyer Cosmetics).

Accordingly, what is needed is a fexofenadine formulation that is paraben free that is bioequivalent to the current suspension formulation of fexofenadine.

### SUMMARY OF THE INVENTION

The present disclosure is directed to a paraben-free aqueous pharmaceutical suspensions, having a pH 5.8 to 7.0, comprising
0.03% to 1.20% (w/w) of fexofenadine zwitterionic dihydrate Form I of formula (I)

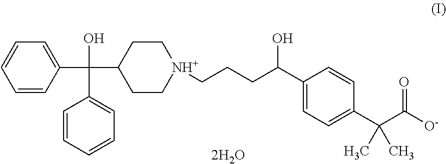 [SMILES: CC(C)(C(=O)[O-])c1ccc(C(O)CCC[NH+]2CCC(C(O)(c3ccccc3)c3ccccc3)CC2)cc1]

0.01% to 0.20% (w/w) of a wetting agent;
a suspending agent comprising 0.10% to 0.50% (w/w) of a hydrocolloid gum,
a sweetener system comprising 5% to 40% (w/w) of sucrose, and
5% to 40% (w/w) of xylitol (in which optionally the ratio of the amount of (sucrose) (xylitol) is 1:1 to 2:1);
a preservative system comprising (i) potassium sorbate, domiphen bromide, cetylpyridinium chloride, or sodium benzoate, and
0.01% to 0.25% (w/w) of edetate disodium;
a buffer system comprising 0.06% to 1.05% (w/w) of sodium phosphate monobasic or a corresponding equivalent amount of a sodium phosphate monobasic hydrate, or 0.06% to 1.05% (w/w) of potassium phosphate monobasic, and
0.32% to 2.69% (w/w) of sodium phosphate dibasic or a corresponding equivalent amount of a sodium phosphate dibasic hydrate, or 0.32% to 2.69% (w/w) of potassium phosphate dibasic or a corresponding equivalent amount of a potassium phosphate dibasic hydrate;
1.0% to 10.0% (w/w) of propylene glycol; and
0.20% to 0.70% (w/w) of a flavoring agent,
   wherein the aqueous pharmaceutical suspension is paraben-free. These aqueous pharmaceutical suspensions are bioequivalent to the existing formulations of fexofenadine.

In certain embodiments, the preservative system comprises potassium sorbate and 0.01% to 0.25% (w/w) of edetate disodium. In certain embodiments, the preservative system comprises domiphen bromide and 0.01% to 0.25% (w/w) of edetate. In certain embodiments, the preservative system comprises disodium Cetylpyridinium chloride and 0.01% to 0.25% (w/w) of edetate. In certain embodiments, the preservative system comprises sodium benzoate and 0.01% to 0.25% (w/w) of edetate.

In particular, the present disclosure is directed to a paraben-free aqueous pharmaceutical suspension, having a pH 5.8 to 7.0, comprising
0.03% to 1.20% (w/w) of fexofenadine zwitterionic dihydrate Form I of formula (I)

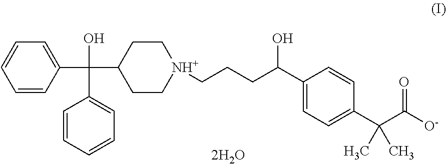 [SMILES: CC(C)(C(=O)[O-])c1ccc(C(O)CCC[NH+]2CCC(C(O)(c3ccccc3)c3ccccc3)CC2)cc1]

0.01% to 0.20% (w/w) of a wetting agent;
a suspending agent comprising 0.10% to 0.50% (w/w) of a hydrocolloid gum,
a sweetener system comprising 5% to 40% (w/w) of sucrose, and
5% to 40% (w/w) of xylitol (in which optionally the ratio of the amount of (sucrose) (xylitol) is 1:1 to 2:1);
a preservative system comprising 0.1% to 2% (w/w) potassium sorbate, and
0.01% to 0.25% (w/w) of edetate disodium;
a buffer system comprising 0.06% to 1.05% (w/w) of sodium phosphate monobasic or a corresponding equivalent amount of a sodium phosphate monobasic hydrate, or 0.06% to 1.05% (w/w) of potassium phosphate monobasic, and
0.32% to 2.69% (w/w) of sodium phosphate dibasic or a corresponding equivalent amount of a sodium phosphate dibasic hydrate, or 0.32% to 2.69% (w/w) of potassium phosphate dibasic or a corresponding equivalent amount of a potassium phosphate dibasic hydrate;
1.0% to 10.0% (w/w) of propylene glycol; and
0.20% to 0.70% (w/w) of a flavoring agent,
   wherein the aqueous pharmaceutical suspension is paraben-free. These aqueous pharmaceutical suspensions are bioequivalent to the existing formulations of fexofenadine.

In certain embodiments, the suspensions comprise 0.03% to 1.20% of fexofenadine zwitterionic dihydrate Form I of formula (I) having a particle size of less than 280 μm for at least 90% of the fexofenadine zwitterionic dihydrate Form I. In other embodiments, the particle size is less than 50 μm for at least 90% of the fexofenadine zwitterionic dihydrate Form I. In other embodiments, the particle size is less than 40 μm for at least 90% of the fexofenadine zwitterionic dihydrate Form I.

In certain embodiments, the fexofenadine zwitterionic dihydrate Form I is 0.3%-0.7% (w/w). In certain embodiments, the fexofenadine zwitterionic dihydrate Form I is 0.4-0.6% (w/w).

In certain embodiments, the wetting agent is 0.01-0.07% (w/w) or 0.02-0.07% (w/w). In certain embodiments, the wetting agent is ionic. In a particular embodiment, the wetting agent is Poloxamer 188. In another particular embodiment, the wetting agent is Poloxamer 407.

In certain embodiments, the hydrocolloid gum is 0.1-0.5% (w/w) or 0.2-0.4% (w/w). In certain embodiment, the hydrocolloid gum is xanthan gum.

In certain embodiments, the xylitol is 7.5-10% (w/w) or 8.5-9.5% (w/w). In certain embodiments, the sucrose is 17.3-18.5% (w/w) or 17.5-18.0% (w/w) or 17.5%-18.5 (w/w).

In certain embodiments, the potassium sorbate is 0.1-0.6% (w/w). In certain embodiments, the potassium sorbate is 0.2-0.5% (w/w)

In certain embodiments, the edetate disodium is 0.05-0.2% (w/w) or 0.10-0.18% (w/w).

In certain embodiments, the buffer system comprises 0.4% to 0.9% (w/w) of sodium phosphate monobasic or a corresponding equivalent amount of a sodium phosphate monobasic hydrate; and 0.5% to 0.8% (w/w) of sodium phosphate dibasic or a corresponding equivalent amount of a sodium phosphate dibasic hydrate.

In certain embodiments, the propylene glycol is 1.8-2.5% (w/w) or 1.9-2.4%.

In certain embodiments, the flavoring agent is 0.2-0.6% (w/w). In certain embodiments, the flavoring agent is artificial raspberry cream flavor or artificial orange cream flavor.

In certain embodiments, the paraben-free aqueous pharmaceutical suspensions of the invention further comprise, 0.07% to 1.1% (w/w) of an opacifying agent. In certain embodiments, the opacifying agent is titanium dioxide.

In certain embodiments, the paraben-free aqueous pharmaceutical suspension of the invention comprises 0.4% to 0.6% (w/w) of fexofenadine zwitterionic dihydrate Form I, 1.9% to 2.4% (w/w) of propylene glycol, 0.15% to 0.5% (w/w) of potassium sorbate, 0.10% to 0.18% (w/w) of edetate disodium, 0.9% to 1.2% (w/w) of sodium phosphate dibasic heptahydrate, 0.5% to 0.8% (w/w) of sodium phosphate monobasic monohydrate, 0.2% to 0.4% (w/w) of xanthan gum, 0.02% to 0.05% (w/w) of Poloxamer 407, 0.08% to 1.0% (w/w) of titanium dioxide, 17.5% to 18.0% (w/w) of sucrose, 8.5 to 9.50% (w/w) of xylitol, and 0.3% to 0.5% (w/w) of a flavoring agent. In further embodiments, 17.5% to 18.5 (w/w) of sucrose are used in the formulation.

In certain embodiments, the paraben-free aqueous pharmaceutical suspension of the invention comprises 0.4% to 0.6% (w/w) of fexofenadine zwitterionic dihydrate Form I, 1.9% to 2.4% (w/w) of propylene glycol, 0.001% to 10.0% (w/w) of domiphen bromide, 0.10% to 0.18% (w/w) of edetate disodium, 0.9% to 1.2% (w/w) of sodium phosphate dibasic heptahydrate, 0.5% to 0.8% (w/w) of sodium phosphate monobasic monohydrate, 0.2% to 0.4% (w/w) of xanthan gum, 0.02% to 0.05% (w/w) of Poloxamer 407, 0.08% to 1.0% (w/w) of titanium dioxide, 17.5% to 18.0% (w/w) of sucrose, 8.5 to 9.50% (w/w) of xylitol, and 0.3% to 0.5% (w/w) of a flavoring agent. In further embodiments, 17.5% to 18.5 (w/w) of sucrose are used in the formulation.

In certain embodiments, the paraben-free aqueous pharmaceutical suspension of the invention comprises 0.4% to 0.6% (w/w) of fexofenadine zwitterionic dihydrate Form I, 1.9% to 2.4% (w/w) of propylene glycol, 0.0001% to 1.000% (w/w) of Cetylpyridinium chloride, 0.10% to 0.18% (w/w) of edetate disodium, 0.9% to 1.2% (w/w) of sodium phosphate dibasic heptahydrate, 0.5% to 0.8% (w/w) of sodium phosphate monobasic monohydrate, 0.2% to 0.4% (w/w) of xanthan gum, 0.02% to 0.05% (w/w) of Poloxamer 407, 0.08% to 1.0% (w/w) of titanium dioxide, 17.5% to 18.0% (w/w) of sucrose, 8.5 to 9.50% (w/w) of xylitol, and 0.3% to 0.5% (w/w) of a flavoring agent. In further embodiments, 17.5% to 18.5 (w/w) of sucrose are used in the formulation.

In certain embodiments, the paraben-free aqueous pharmaceutical suspension of the invention comprises 0.4% to 0.6% (w/w) of fexofenadine zwitterionic dihydrate Form I, 1.9% to 2.4% (w/w) of propylene glycol, 0.001% to 10.0% (w/w) of sodium benzoate, 0.10% to 0.18% (w/w) of edetate disodium, 0.9% to 1.2% (w/w) of sodium phosphate dibasic heptahydrate, 0.5% to 0.8% (w/w) of sodium phosphate monobasic monohydrate, 0.2% to 0.4% (w/w) of xanthan gum, 0.02% to 0.05% (w/w) of Poloxamer 407, 0.08% to 1.0% (w/w) of titanium dioxide, 17.5% to 18.0% (w/w) of sucrose, 8.5 to 9.50% (w/w) of xylitol, and 0.3% to 0.5% (w/w) of a flavoring agent. In further embodiments, 17.5% to 18.5 (w/w) of sucrose are used in the formulation.

Also provided are methods of preparing the paraben-free aqueous pharmaceutical suspensions of the invention.

Other features and advantages of the invention will be apparent from the detailed description and the examples that follow.

### BRIEF DESCRIPTION OF THE DRAWINGS

The foregoing summary, as well as the following detailed description of the invention, will be better understood when read in conjunction with the appended FIGURE. For the purpose of illustrating the invention, the FIGURE demonstrates embodiments of the present invention. It should be understood, however, that the invention is not limited to the precise arrangements, examples, and instrumentalities shown.

FIG. 1 shows conversion from Fexofenadine Form I anhydrous to Form I Zwitterion dihydrate.

### DETAILED DESCRIPTION

This disclosure is based on the discovery that it is possible to generate fexofenadine liquid suspension formulations that lack paraben. These formulations avoid the safety concern recently associated with these substances without changing critical quality attributes and bioequivalency of the current product. The disclosure overcomes the challenges associated with reformulating a suspension in which stability is sensible to changes and the API Fexofenadine, which has low pH-dependent solubility. Accordingly, this disclosure provides a preservative system without changing any critical product features of the existing fexofenadine suspension formulations.

This disclosure is based on the discovery that despite the synergism between paraben and EDTA is it is possible to generate paraben free fexofenadine liquid suspension formulations by replacing paraben with domiphen bromide, Cetylpyridinium chloride, sodium benzoate or potassium sorbate.

In particular, this disclosure is based on the discovery that potassium sorbate has an equivalent preservative performance of parabens, keeping the physical and chemical features of an incredibly challenging API (fexofenadine) and the pH range of the formulation.

Accordingly, the fexofenadine liquid suspensions of the current embodiments which lack parabens (i.e., are paraben-free) are bioequivalent to the currently marketed fexofenadine suspension formulations. For clarity of disclosure, and not by way of limitation, the detailed description of the invention is divided into subsections that describe or illustrate certain features, embodiments, or applications of the present invention.

### Definitions

As used herein, the term “about” when referring to a measurable value such as an amount, a temporal duration, and the like, is meant to encompass variations of ±0.20% or ±10%, more preferably ±5%, even more preferably ±1%, and still more preferably ±0.1% from the specified value, as such variations are appropriate to perform the disclosed methods.

As used herein, the singular forms “a,” “an,” and “the” include the plural reference. Reference to a particular numerical value includes at least that particular value unless the context clearly indicates otherwise. Thus, for example, reference to “a substance” is a reference to at least one of such substances and equivalents thereof known to those skilled in the art, and so forth.

When a value is expressed as an approximation by use of “about,” it will be understood that the particular value forms another embodiment. In general, use of “about” indicates approximations that can vary depending on the desired properties sought to be obtained by the disclosed subject matter and is to be interpreted in the specific context in which it is used. The person skilled in the art will be able to interpret this as a matter of routine. In some instances, the number of significant FIGURES used for a particular value may be one non-limiting method of determining the extent of the word “about.” In other cases, the gradations used in a series of values may be used to determine the intended range available to the term “about” for each value. Where present, all ranges are inclusive and combinable. That is, references to values stated in ranges include every value within that range.

As used herein, while data is given with full disclosure of all significant FIGURES, those of skilled in the art would understand that the data can also be understood when rounded to 1 or 2 decimal places.

When a list is presented, unless stated otherwise, it is to be understood that each individual element of that list and every combination of that list is to be interpreted as a separate embodiment. For example, a list of embodiments presented as “A, B, or C” is to be interpreted as including the embodiments “A,” “B,” “C,” “A or B,” “A or C,” “B or C,” or “A, B, or C.”

It is to be appreciated that certain features of the invention which are, for clarity, described herein in the context of separate embodiments, may also be provided in combination in a single embodiment. That is, unless obviously incompatible or excluded, each individual embodiment is deemed to be combinable with any other embodiment(s) and such a combination is considered to be another embodiment. Conversely, various features of the invention that are, for brevity, described in the context of a single embodiment, may also be provided separately or in any sub-combination. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Finally, while an embodiment may be described as part of a series of steps or part of a more general structure, each said step may also be considered an independent embodiment in itself. Before certain embodiments are described in greater detail, it is to be understood that this invention is not limited to certain embodiments described, and as such may, of course, vary. It is also to be understood that the terminology used herein is for the purpose of describing certain embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods, and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates, which may need to be independently confirmed.

The terms “patient” or “subject” as used herein are interchangeable and refer to a mammalian animal. In some embodiments, the patient or subject is a human. In other embodiments, the patient or subject is a veterinary or farm animal, a domestic animal or pet, or animal normally used for clinical research. In some embodiments, the patient is an adult, i.e., 18 years of age or older. In other embodiments, the patient is a child, i.e., under the age of 18.

The term “treating” includes ameliorating a disease or disorder (i.e., arresting or reducing the development of the disease or at least one of the clinical symptoms thereof). In some embodiments, “treating” refers to ameliorating at least one physical parameter, which may not be discernible by the subject. In other embodiments, “treating” refers to modulating the disease or disorder, either physically, (e.g., stabilizing a discernible symptom), physiologically, (e.g., stabilizing a physical parameter), or both. In further embodiments, “treating” refers to delaying the onset of the disease or disorder.

The terms “fexofenadine zwitterion” and “fexofenadine zwitterion dihydrate” are used interchangeably and refer to 4-[1-hydroxy-4-[4-(hydroxydiphenylmethyl)-1-piperidinyl]-butyl]-α,α-dimethyl benzeneacetic acid dihydrate. Fexofenadine zwitterion dihydrate has the following structure:

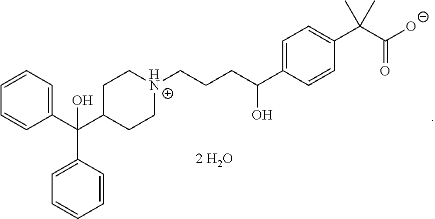

“Buffer system” is used to adjust the pH of the suspension to minimize the solubility of the constituent fexofenadine and to maintain that fexofenadine as fexofenadine zwitterionic dihydrate Form I for a minimum of about 18 months; more particularly for at least about 24 months. Examples of the buffer system include (citric acid/sodium phosphate dibasic or sodium phosphate dibasic hydrate) system, (succinic acid/sodium hydroxide) system, (citric acid/sodium citrate, sodium citrate hydrate or potassium citrate) system, (maleic acid/sodium hydroxide) system, (fumaric acid/sodium hydroxide) system, (sodium phosphate monobasic, sodium phosphate monobasic hydrate or potassium phosphate monobasic/sodium phosphate dibasic, sodium phosphate dibasic hydrate, potassium phosphate dibasic or potassium phosphate dibasic hydrate) system, particularly (sodium phosphate monobasic, sodium phosphate monobasic hydrate or potassium phosphate monobasic/sodium phosphate dibasic, sodium phosphate dibasic hydrate, potassium phosphate dibasic or potassium phosphate dibasic hydrate) system, more particularly (sodium phosphate monobasic, or sodium phosphate monobasic hydrate/sodium phosphate dibasic, or sodium phosphate dibasic hydrate) system, even more particularly (sodium phosphate monobasic monohydrate/sodium phosphate dibasic heptahydrate) system.

“Potassium phosphate monobasic” means KH₂PO₄. “Potassium phosphate dibasic” means K₂HPO₄.

“Potassium phosphate dibasic hydrate” includes, for example, potassium phosphate dibasic trihydrate and potassium phosphate dibasic hexahydrate.

“Sodium phosphate monobasic” means NaH₂PO₄.

“Sodium phosphate monobasic hydrate” includes, for example, sodium phosphate monobasic monohydrate, and sodium phosphate monobasic dihydrate.

“Sodium phosphate dibasic” means Na₂HPO₄.

“Sodium phosphate dibasic hydrate” includes, for example, sodium phosphate dibasic dihydrate, sodium phosphate dibasic heptahydrate, and sodium phosphate dibasic dodecahydrate.

“Poloxamer” is α-hydro-ω-hydroxypoly(oxyethylene)poly(oxypropylene) poly(oxyethylene) block copolymer. Examples of Poloxamer include Poloxamer 407 and Poloxamer 188.

“Particle size” is determined utilizing Low-Angle Laser Light-Scattering (LALLS), and is calculated as spheres of equivalent diameter to the test sample. Particle size distribution is described as the volume % above or below the stated diameter. For example, the Dv₁₀, Dv₅₀, and Dv₉₀ correspond, respectively, to the particle diameter as which 10, 50, and 90% of the total particle size distribution volume is below the stated diameter.

### Paraben-Free Aqueous Fexofenadine Suspensions

It is an object of the present invention to provide a paraben-free aqueous pharmaceutical suspension containing fexofenadine zwitterionic dihydrate Form I, with no impact on the quality and safety of the aqueous composition while maintaining the zwitterion form of Fexofenadine.

In a general aspect the invention relates to a paraben-free aqueous pharmaceutical suspension, comprising fexofenadine zwitterionic dihydrate Form I of formula (I):

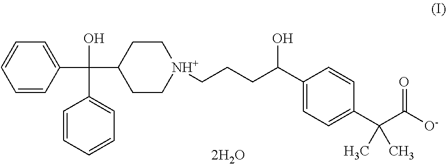 [SMILES: CC(C)(C(=O)[O-])c1ccc(C(O)CCC[NH+]2CCC(C(O)(c3ccccc3)c3ccccc3)CC2)cc1]

a wetting agent;
a suspending agent,
a sweetener system;
a preservative system;
a buffer system; and
a flavoring agent, wherein the aqueous pharmaceutical suspension is paraben-free.

The aqueous pharmaceutical suspension formulation can optionally contain an opacifying agent, such as for example, titanium oxide. As used herein, an aqueous pharmaceutical suspension is “paraben-free” or “free of paraben” when the formulation does not comprise or contain any paraben (i.e., the formulation lacks paraben).

One particular embodiment of the invention is the paraben-free aqueous pharmaceutical suspension, wherein the pH is about 5.00 to about 8.00; or more particularly 5.80 to about 7.00.

Another embodiment of the invention is the paraben-free aqueous pharmaceutical suspension, wherein the particle size is less than about 50 μm; or more particularly 40 μm; for at least about 90% of the fexofenadine zwitterionic dihydrate Form I.

Another particular embodiment of the invention is the paraben-free aqueous pharmaceutical suspension, wherein the fexofenadine zwitterionic dihydrate Form I is about 0.03%-1.20% (w/w); more particularly about 0.3-0.7% (w/w), further more particularly about 0.4-0.6% (w/w).

Another embodiment of the invention is the paraben-free aqueous pharmaceutical suspension, wherein the wetting agent is about 0.01% to about 0.07% (w/w); more particularly about 0.02% to about 0.05% (w/w).

Another particular embodiment of the invention is the paraben-free aqueous pharmaceutical suspension, wherein the wetting agent is nonionic, such as Poloxamer 407 and Poloxamer 188. In certain embodiments, the wetting agent is Poloxamer 407.

Another particular embodiment of the invention is the paraben-free aqueous pharmaceutical suspension, wherein the suspending agent is a hydrocolloid gum.

Another embodiment of the invention is the paraben-free aqueous pharmaceutical suspension, wherein the hydrocolloid gum is, about 0.1% to about 0.5% (w/w); more particularly about 0.2% to about 0.4% (w/w).

Another particular embodiment of the invention is the paraben-free aqueous pharmaceutical suspension, wherein the hydrocolloid gum is xanthan gum.

Another particular embodiment of the invention is the paraben-free aqueous pharmaceutical suspension, wherein the sweetener system contains one or more of xylitol, sorbitol, maltitol, sucrose, or inverted sucrose.

Another particular embodiment of the invention is the paraben-free aqueous pharmaceutical suspension, wherein xylitol, sorbitol or sorbitol solution, or maltitol solution is, 0% to about 20% (w/w); more particularly about 10% to about 20% (w/w); more particularly about 7.5% to about 10% (w/w); more particularly about 8.5% to about 9.5% (w/w).

Another particular embodiment of the invention is the paraben-free aqueous pharmaceutical suspension, wherein sucrose or invert sucrose is, about 10% to about 40% (w/w); more particularly about 10% to about 20% (w/w); more particularly about 17.3% to about 18.5% (w/w); more particularly about 17.5% to about 18.0% (w/w); more particularly about 17.5% to about 18.5% (w/w).

Another particular embodiment of the invention is the paraben-free aqueous pharmaceutical suspension, wherein the sweetener system comprises xylitol and sucrose.

Another particular embodiment of the invention is the paraben-free aqueous pharmaceutical suspension, wherein the sweetener system comprises xylitol and sucrose.

Another particular embodiment of the invention is the paraben-free aqueous pharmaceutical suspension, wherein the sweetener system comprises xylitol and sucrose, and the ratio of the amount of sucrose:xylitol is about 2:1.

Another particular embodiment of the invention is the paraben-free aqueous pharmaceutical suspension, wherein the sweetener system comprises sucralose and sucrose.

Another particular embodiment of the invention is the paraben-free aqueous pharmaceutical suspension, wherein the sweetener system comprises about 7.5% to about 10% (w/w) of xylitol, and about 17.3% to about 18.5% (w/w) of sucrose.

Another particular embodiment of the invention is the paraben-free aqueous pharmaceutical suspension, wherein the sweetener system comprises about 8.5% to about 9.50% (w/w) of xylitol, and about 17.5% to about 18.0% (w/w) of sucrose.

Another particular embodiment of the invention is the paraben-free aqueous pharmaceutical suspension, wherein the sweetener system comprises about 8.5% to about 9.50% (w/w) of xylitol, and about 17.5% to about 18.5% (w/w) of sucrose.

Another particular embodiment of the invention is the paraben-free aqueous pharmaceutical suspension, wherein the ratio of the amount of (sucrose or invert sucrose):(xylitol, sorbitol or sorbitol solution, or maltitol solution) is about 1:1 to 2:1.

Another particular embodiment of the invention is the paraben-free aqueous pharmaceutical suspension, wherein the ratio of the amount of (sucrose or invert sucrose):(xylitol, sorbitol or sorbitol solution, or maltitol solution) is about 2:1.

Another particular embodiment of the invention is the paraben-free aqueous pharmaceutical suspension, wherein the ratio of the amount of (sucrose or invert sucrose):(xylitol, sorbitol or sorbitol solution, or maltitol solution) is about 1:1.

One particular embodiment of the invention is the paraben-free aqueous pharmaceutical suspension wherein the preservative system comprises potassium sorbate and edetate disodium.

Another particular embodiment of the invention is the paraben-free aqueous pharmaceutical suspension wherein the preservative system comprises domiphen bromide and edetate disodium.

Another particular embodiment of the invention is the paraben-free aqueous pharmaceutical suspension wherein the preservative system comprises cetylpyridinium chloride and edetate disodium.

Another particular embodiment of the invention is the paraben-free aqueous pharmaceutical suspension wherein the preservative system comprises sodium benzoate and edetate disodium.

Another particular embodiment of the invention is the paraben-free aqueous pharmaceutical suspension wherein the preservative system comprises edetate disodium and one or more of domiphen bromide, cetylpyridinium chloride, sodium benzoate, or potassium sorbate.

Another particular embodiment of the invention is the paraben-free aqueous pharmaceutical suspension wherein the preservative system contains only edetate disodium and one or more of domiphen bromide, cetylpyridinium chloride, sodium benzoate, or potassium sorbate.

Another particular embodiment of the invention is the paraben-free aqueous pharmaceutical suspension wherein the preservative system contains only edetate disodium and domiphen bromide.

Another particular embodiment of the invention is the paraben-free aqueous pharmaceutical suspension wherein the preservative system contains only edetate disodium and cetylpyridinium chloride.

Another particular embodiment of the invention is the paraben-free aqueous pharmaceutical suspension wherein the preservative system contains only edetate disodium and sodium benzoate.

Another particular embodiment of the invention is the paraben-free aqueous pharmaceutical suspension wherein the preservative system contains only edetate disodium and potassium sorbate.

Another particular embodiment of the invention is the paraben-free aqueous pharmaceutical suspension, wherein the preservative system comprises about 0.1% to about 2% (w/w) of potassium sorbate, alternatively about 0.1% to about 4% (w/w), alternatively about 0.35% to about 4% (w/w). alternatively about 0.35% to about 2% (w/w), alternatively about 0.3% to about 1.5% (w/w), alternatively about 0.3% to about 2% (w/w), alternatively about 0.25% to 0.6% (w/w), alternatively about 0.35% to 0.6% (w/w), alternatively about 0.36% to 0.6% (w/w), alternatively above 0.3% to about 1% (w/w), alternatively 0.3% to about 0.7 (w/w), alternatively from at least 0.4% to about 2% (w/w), particularly about 0.1% to about 0.6% (w/w), further more particularly about 0.15 to about 0.5% (w/w).

Another particular embodiment of the invention is the paraben-free aqueous pharmaceutical suspension, wherein the preservative system comprises about 0.0001% to about 10% of domiphen bromide, particularly 0.0001% to about 5% (w/w), further more particularly about 0.001 to about 1.0% (w/w).

Another particular embodiment of the invention is the paraben-free aqueous pharmaceutical suspension, wherein the preservative system comprises about 0.0001% to about 1% of cetylpyridinium chloride, particularly about 0.0001% to about 0.6% (w/w), further more particularly about 0.005 to about 0.01% (w/w).

Another particular embodiment of the invention is the paraben-free aqueous pharmaceutical suspension, wherein the preservative system comprises about 0.001% to about 10% of sodium benzoate, particularly about 0.01% to about 1% (w/w), further more particularly about 0.3 to about 0.8% (w/w).

Another particular embodiment of the invention is the paraben-free aqueous pharmaceutical suspension, wherein the edetate disodium is about 0.01% to about 0.25% (w/w), particularly about 0.05-0.2% (w/w) further more particularly about 0.10% to 0.18% (w/w).

Another particular embodiment of the invention is the paraben-free aqueous pharmaceutical suspension, wherein the buffer system comprises sodium phosphate monobasic monohydrate and sodium phosphate dibasic heptahydrate.

Another particular embodiment of the invention is the paraben-free aqueous pharmaceutical suspension, wherein the buffer system only contains sodium phosphate monobasic monohydrate and sodium phosphate dibasic heptahydrate.

Another particular embodiment of the invention is the paraben-free aqueous pharmaceutical suspension, wherein the buffering uses a buffer system comprising:
about 0.06% to about 1.05% (w/w) of sodium phosphate monobasic or corresponding equivalent amount of a sodium phosphate monobasic hydrate, or about 0.069% to about 1.190% (w/w) of potassium phosphate monobasic; and
about 0.32% to about 2.69% (w/w) of sodium phosphate dibasic or corresponding equivalent amount of a sodium phosphate dibasic hydrate, or about 0.39% to about 3.30% (w/w) of potassium phosphate dibasic or corresponding equivalent amount of a potassium phosphate dibasic hydrate.

Another particular embodiment of the invention is the paraben-free aqueous pharmaceutical suspension, wherein the buffering uses a buffer system comprising:
about 0.22% to about 0.87% (w/w) of sodium phosphate monobasic or a corresponding equivalent amount of a sodium phosphate monobasic hydrate, or about 0.25% to about 0.99% (w/w) of potassium phosphate monobasic; and
about 0.32% to about 1.15% (w/w) of sodium phosphate dibasic or a corresponding equivalent amount of sodium phosphate dibasic hydrate, or about 0.39% to about 1.41% (w/w) of potassium phosphate dibasic or a corresponding equivalent amount of potassium phosphate dibasic hydrate.

Another particular embodiment of the invention is the paraben-free aqueous pharmaceutical suspension, wherein the buffering uses a buffer system comprising,
about 0.06% to about 1.05% (w/w) of sodium phosphate monobasic or a corresponding equivalent amount of a sodium phosphate monobasic hydrate; and
about 0.32% to about 2.69% (w/w) of sodium phosphate dibasic or a corresponding amount of a sodium phosphate dibasic hydrate.

Another particular embodiment of the invention is the paraben-free aqueous pharmaceutical suspension, wherein the buffering uses a buffer system comprising, about 0.4% to about 0.9% (w/w) of sodium phosphate monobasic monohydrate, and about 0.8% to about 1.3% (w/w) of sodium phosphate dibasic heptahydrate.

Another particular embodiment of the invention is the paraben-free aqueous pharmaceutical suspension, wherein the buffering uses a buffer system comprising, about 0.5% to about 0.8% (w/w) of sodium phosphate monobasic monohydrate, and about 0.9% to about 1.2% (w/w) of sodium phosphate dibasic heptahydrate.

Another particular embodiment of the invention is the paraben-free aqueous pharmaceutical suspension, wherein the paraben-free aqueous pharmaceutical suspension further comprises as a co-solvent.

Another particular embodiment of the invention is the paraben-free aqueous pharmaceutical suspension, wherein the paraben-free aqueous pharmaceutical suspension further comprises propylene glycol as a co-solvent.

Another particular embodiment of the invention is the paraben-free aqueous pharmaceutical suspension, comprising, about 0.1% to about 15.0% (w/w); more particularly about 1.0% to about 10.0%; or more particularly about 1.8% to about 2.5% (w/w); or further more particularly about 1.9% to about 2.4% (w/w) of a co-solvent, such as propylene glycol.

Another particular embodiment of the invention is the paraben-free aqueous pharmaceutical suspension, comprising, 0% to about 4% (w/w); more particularly 0% to about 2% (w/w); or further more particularly 0% to about 1% (w/w) of a co-solvent, such as polyethylene glycol 200, polyethylene glycol 300 or polyethylene glycol 400.

Another particular embodiment of the invention is the paraben-free aqueous pharmaceutical suspension, optionally further comprising opacifying agent. Yet another particular embodiment of the invention is the paraben-free aqueous pharmaceutical suspension, optionally further comprising titanium dioxide.

Another particular embodiment of the invention is the paraben-free aqueous pharmaceutical suspension, optionally further comprising, 0% to about 2% (w/w); more particularly about 0.07% to about 1.1%; or further more particularly about 0.08% to about 1.0% (w/w) of an opacifying agent, such as titanium dioxide.

Another particular embodiment of the invention is the paraben-free aqueous pharmaceutical suspension, comprising, about 0.20% to about 0.70% (w/w); more particularly about 0.2% to about 0.6% (w/w); or further more particularly about 0.3% to about 0.5% (w/w) of a flavoring agent, such as artificial raspberry cream flavor or artificial orange cream flavor.

Another particular embodiment of the invention is the paraben-free aqueous pharmaceutical suspension, comprising artificial raspberry cream flavor, artificial vanilla, and/or artificial orange cream flavor as the favoring agent.

Another particular embodiment of the invention is a paraben-free aqueous pharmaceutical suspension, having a pH 5.8 to 7.0, comprising:
fexofenadine zwitterionic dihydrate Form I of formula (I)

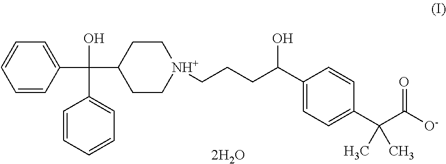 [SMILES: CC(C)(C(=O)[O-])c1ccc(C(O)CCC[NH+]2CCC(C(O)(c3ccccc3)c3ccccc3)CC2)cc1]

a wetting agent;
a suspending agent comprising a hydrocolloid gum,
a sweetener system comprising sucrose, and xylitol;
a preservative system comprising potassium sorbate and edetate disodium;
a buffer system comprising sodium phosphate monobasic or a corresponding equivalent amount of a sodium phosphate monobasic hydrate, or potassium phosphate monobasic, and sodium phosphate dibasic or a corresponding equivalent amount of a sodium phosphate dibasic hydrate, or potassium phosphate dibasic or a corresponding equivalent amount of a potassium phosphate dibasic hydrate;
propylene glycol; and
a flavoring agent,
wherein the aqueous pharmaceutical suspension is paraben-free. In certain embodiments of this suspension, the ratio of the amount of (sucrose):(xylitol) is about 1:1 to about 2:1. This embodiment of the paraben-free aqueous pharmaceutical suspension may have any of components in the amounts described above. In another embodiment of this embodiment of the paraben-free aqueous pharmaceutical suspension, the preservative system contains only potassium sorbate and edetate disodium.

Another particular embodiment of the invention is a paraben-free aqueous pharmaceutical suspension, having a pH 5.8 to 7.0, comprising:
fexofenadine zwitterionic dihydrate Form I of formula (I)

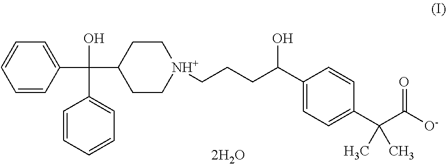 [SMILES: CC(C)(C(=O)[O-])c1ccc(C(O)CCC[NH+]2CCC(C(O)(c3ccccc3)c3ccccc3)CC2)cc1]

a wetting agent;
a suspending agent comprising a hydrocolloid gum,
a sweetener system comprising sucrose, and xylitol;
a preservative system comprising edetate disodium and one of (i) domiphen bromide, (ii) cetylpyridinium chloride and (iii) sodium benzoate;
a buffer system comprising sodium phosphate monobasic or a corresponding equivalent amount of a sodium phosphate monobasic hydrate, or potassium phosphate monobasic, and sodium phosphate dibasic or a corresponding equivalent amount of a sodium phosphate dibasic hydrate, or potassium phosphate dibasic or a corresponding equivalent amount of a potassium phosphate dibasic hydrate;
propylene glycol; and
a flavoring agent,
wherein the aqueous pharmaceutical suspension is paraben-free. In certain embodiments of this suspension, the ratio of the amount of (sucrose):(xylitol) is about 1:1 to about 2:1. This embodiment of the paraben-free aqueous pharmaceutical suspension may have any of components in the amounts described above. In another embodiment of this embodiment of the paraben-free aqueous pharmaceutical suspension, the preservative system contains only edetate disodium and domiphen bromide. In another embodiment of this embodiment of the paraben-free aqueous pharmaceutical suspension, the preservative system contains only edetate disodium and cetylpyridinium chloride. In another embodiment of this embodiment of the paraben-free aqueous pharmaceutical suspension, the preservative system contains only edetate disodium and sodium benzoate.

In certain embodiments of the disclosure, the paraben-free aqueous formulation comprises Formulation A1 to A8 (shown in Table A below). In certain embodiments of Formulation A1 to A8, the flavoring agent is artificial raspberry cream flavor. Titanium oxide may be omitted in certain embodiments of Formulation A1 to A8.

TABLE A

| | Formulation A1 | Formulation A2 | |
|---|---|---|---|
| Components | Percentage (w/w) | Percentage (w/w) | Function |
| Fexofenadine zwitterionic | 0.3-0.7% | 0.4-0.6% | Active substance |
| hydrate Form I | | | |
| Propylene Glycol | 1.8-2.5% | 1.9-2.4% | Co-Solvent |
| Edetate Disodium dihydrate | 0.05-0.2% | 0.10-0.18% | Preservative Aid |
| Potassium sorbate | 0.1-0.6% | 0.15-0.5%; 0.35- | Preservative |
| | | 0.6%, or 0.3-0.6% | |
| Xanthan Gum | 0.1-0.5% | 0.2-0.4% | Suspending Agent |
| Poloxamer 407 | 0.01-0.07% | 0.02-0.05% | Wetting Agent |
| Titanium Dioxide | 0.07-1.1% | 0.08-1.0% | Opacifier |
| Sodium Phosphate Monobasic | 0.4-0.9% | 0.5-0.8% | Buffer |
| Monohydrate | | | |
| Sodium Phosphate Dibasic | 0.8-1.3% | 0.9-1.2% | Buffer |
| Heptahydrate | | | |
| Flavoring agent | 0.2-0.6% | 0.3-0.5% | Flavor |
| Sucrose | 17.3-18.5% | 17.5-18.0% or | Sweetener |
| | | 17.5-18.5% | |
| Xylitol | 7.5-10% | 8.5-9.50% | Sweetener |
| Purified Water | q.s. | q.s. | Primary solvent |

| | Formulation A3 | Formulation A4 | |
|---|---|---|---|
| Components | Percentage (w/w) | Percentage (w/w) | Function |
| Fexofenadine zwitterionic | 0.3-0.7% | 0.4-0.6% | Active substance |
| hydrate Form I | | | |
| Propylene Glycol | 1.8-2.5% | 1.9-2.4% | Co-Solvent |
| Edetate Disodium dihydrate | 0.05-0.2% | 0.10-0.18% | Preservative Aid |
| Domiphen Bromide | 0.0001-10% | 0.001-1.0% | Preservative |
| Xanthan Gum | 0.1-0.5% | 0.2-0.4% | Suspending Agent |
| Poloxamer 407 | 0.01-0.07% | 0.02-0.05% | Wetting Agent |
| Titanium Dioxide | 0.07-1.1% | 0.08-1.0% | Opacifier |
| Sodium Phosphate Monobasic | 0.4-0.9% | 0.5-0.8% | Buffer |
| Monohydrate | | | |
| Sodium Phosphate Dibasic | 0.8-1.3% | 0.9-1.2% | Buffer |
| Heptahydrate | | | |
| Flavoring agent | 0.2-0.6% | 0.3-0.5% | Flavor |
| Sucrose | 17.3-18.5% | 17.5-18.0% or | Sweetener |
| | | 17.5-18.5% | |
| Xylitol | 7.5-10% | 8.5-9.50% | Sweetener |
| Purified Water | q.s. | q.s. | Primary solvent |

| | Formulation A5 | Formulation A6 | |
|---|---|---|---|
| Components | Percentage (w/w) | Percentage (w/w) | Function |
| Fexofenadine zwitterionic | 0.3-0.7% | 0.4-0.6% | Active substance |
| hydrate Form I | | | |
| Propylene Glycol | 1.8-2.5% | 1.9-2.4% | Co-Solvent |
| Edetate Disodium dihydrate | 0.05-0.2% | 0.10-0.18% | Preservative Aid |
| Cetylpyridium chloride | 0.0001-1% | 0.0005-0.01% | Preservative |
| Xanthan Gum | 0.1-0.5% | 0.2-0.4% | Suspending Agent |
| Poloxamer 407 | 0.01-0.07% | 0.02-0.05% | Wetting Agent |
| Titanium Dioxide | 0.07-1.1% | 0.08-1.0% | Opacifier |
| Sodium Phosphate Monobasic | 0.4-0.9% | 0.5-0.8% | Buffer |
| Monohydrate | | | |
| Sodium Phosphate Dibasic | 0.8-1.3% | 0.9-1.2% | Buffer |
| Heptahydrate | | | |
| Flavoring agent | 0.2-0.6% | 0.3-0.5% | Flavor |
| Sucrose | 17.3-18.5% | 17.5-18.0% or | Sweetener |
| | | 17.5-18.5% | |
| Xylitol | 7.5-10% | 8.5-9.50% | Sweetener |
| Purified Water | q.s. | q.s. | Primary solvent |

| | Formulation A7 | Formulation A8 | |
|---|---|---|---|
| Components | Percentage (w/w) | Percentage (w/w) | Function |
| Fexofenadine zwitterionic | 0.3-0.7% | 0.4-0.6% | Active substance |
| hydrate Form I | | | |
| Propylene Glycol | 1.8-2.5% | 1.9-2.4% | Co-Solvent |
| Edetate Disodium dihydrate | 0.05-0.2% | 0.10-0.18% | Preservative Aid |
| Sodium Benzoate | 0.0010%- | 0.03-0.8% | Preservative |
| | 10.00% | | |
| Xanthan Gum | 0.1-0.5% | 0.2-0.4% | Suspending Agent |
| Poloxamer 407 | 0.01-0.07% | 0.02-0.05% | Wetting Agent |
| Titanium Dioxide | 0.07-1.1% | 0.08-1.0% | Opacifier |
| Sodium Phosphate Monobasic | 0.4-0.9% | 0.5-0.8% | Buffer |
| Monohydrate | | | |
| Sodium Phosphate Dibasic | 0.8-1.3% | 0.9-1.2% | Buffer |
| Heptahydrate | | | |
| Flavoring agent | 0.2-0.6% | 0.3-0.5% | Flavor |
| Sucrose | 17.3-18.5% | 17.5-18.0% or | Sweetener |
| | | 17.5-18.5% | |
| Xylitol | 7.5-10% | 8.5-9.50% | Sweetener |
| Purified Water | q.s. | q.s. | Primary solvent |

Also provided is a kit comprising a bottle or vial containing the paraben-free aqueous pharmaceutical suspension according to the invention and a syringe or cup.

### Methods of Treatment

The paraben-free aqueous pharmaceutical suspension described herein are useful in relieving symptoms due to an allergy in a patient in need thereof. The methods include administering a therapeutically effective amount of the paraben-free aqueous pharmaceutical suspension to the patient. Another embodiment of the disclosure is use of the paraben-free aqueous pharmaceutical suspension described herein in the treatment of allergy.

One of skill in the would be able to determine suitable allergies that may be treated using the described compositions and dosage forms. For example, the allergies may be due to indoor or outdoor allergens. In some embodiments, the allergy is due to one or more indoor allergens. A variety of indoor allergens are known and include dust mites, a pet allergen, or mold. In further embodiments, the allergy is due to dust mites. In other embodiments, the allergy is due to a pet allergen, e.g., such as in animal saliva, animal urine or animal dander. In still further embodiments, the allergy is due to indoor mold. In yet other embodiments, the allergy is due to one or more outdoor allergens. A number of outdoor allergies are known in the part and include, without limitation, pollen, and mold. In some embodiments, the outdoor allergy is pollen such as from grass, weeds, or trees. In other embodiments, the outdoor allergy is from outdoor mold.

The allergy may result in any number of symptoms in the patient. For examples, the patient may develop one or more of red eye, itchy eye, watery eye, itchy nose, runny nose, stuffy nose, sneezing, nasal congestion, wheezing, coughing, chest tightness, facial pain, rash, hives, shortness of breath, cough, postnasal drip, itchy throat, dry skin, sinus pressure, decreased sense of smell, decreased sense of taste, or poor sleep quality. In some embodiments, the symptom is red eye. In other embodiments, the symptom is itchy eye. In further embodiments, the symptom is watery eye. In yet other embodiments, the symptom is an itchy nose. In still further embodiments, the symptom is a runny nose. In other embodiments, the symptom is a stuffy nose. In further embodiments, the symptom is sneezing. In still other embodiments, the symptom is nasal congestion. In yet further embodiments, the symptom is wheezing. In other embodiments, the symptom is coughing. In further embodiments, the symptom is chest tightness. In yet other embodiments, the symptom is facial pain. In still further embodiments, the symptom is rash. In other embodiments, the symptom is hives. In further embodiments, the symptom is shortness of breath. In yet other embodiments, the symptom is a cough. In still further embodiments, the symptom is postnasal drip. In other embodiments, the symptom is an itchy throat. In further embodiments, the symptom is dry skin. In still other embodiments, the symptom is sinus pressure. In yet further embodiments, the symptom is decreased sense of smell. In other embodiments, the symptom is decreased sense of taste. In further embodiments, the symptom is poor sleep quality. Thus, the methods described herein are useful in treating these symptoms. In some embodiments, the methods ameliorate one or more, or all, of the symptoms. In other embodiments, the methods reduce the number of symptoms in the patient. In further embodiments, the methods prevent the onset of one or more symptoms in the patient.

The liquid compositions and dosage forms are, thus, useful in relieving symptoms due to an upper respiratory allergy in a patient in need thereof. The methods include administering a therapeutically effective amount of the spray-dried formulation or oral solid dosage described herein to the patient. In some embodiments, the symptom is a runny nose, itchy, watery eye, sneezing, itching of the nose, itching of the throat, or a combination thereof. In further embodiments, the upper respiratory allergy is hay fever.

### Preparation of Paraben-Free Suspensions

The paraben-free aqueous pharmaceutical suspensions of the invention can be prepared by adding pre-dissolved components of the sweetener system in purified water. Then adding a dispersion of the suspending agent in a suitable co-solvent to an aliquot of water, previously heated at approximately 25-80° C., particularly 35-80° C., more particularly 35-45° C. The addition of the dispersion using this method promotes hydration and dissolution of the suspending agent. The temperature is maintained through the subsequent addition of a portion of the buffer system (to maintain pH control). The preservative system is then added resulting in the formation of a bulk solution.

The active agent is dispersed in an aqueous solution of the remaining components of the buffer system and the wetting agent. The pH of the solution is controlled prior to addition of the active agent to maintain the appropriate physical form. If added, the opacifying agent is subsequently added and the active dispersion is added to the aforementioned bulk solution previously cooled to 20-35° C., particularly 20-30° C., resulting in the formation of a suspension. The flavoring agent and remaining water, if necessary, are added to the desired weight. The bulk suspension is subsequently milled and de-aerated. The suspension can be prepared by conventional processing equipment.

In some embodiments, the process described generates suspension formulations having the features of Formulation B1 to B8 (shown in Table B below). In one embodiment of Formulation B1 to B8, the flavoring agent is artificial raspberry cream flavor. Titanium oxide may be omitted in certain embodiments of Formulation B1 to B8.

TABLE B

| | Formulation B1 | Formulation B2 | |
|---|---|---|---|
| Components | Percentage (w/w) | Percentage (w/w) | Function |
| Fexofenadine zwitterionic | 0.3-0.7% | 0.4-0.6% | Active substance |
| hydrate Form I | | | |
| Propylene Glycol | 1.8-2.5% | 1.9-2.4% | Co-Solvent |
| Edetate Disodium dihydrate | 0.05-0.2% | 0.10-0.18% | Preservative Aid |
| Potassium sorbate | 0.1-0.6% | 0.15-0.5% or | Preservative |
| | | 0.3-0.6% | |
| Xanthan Gum | 0.1-0.5% | 0.2-0.4% | Suspending Agent |
| Poloxamer 407 | 0.01-0.07% | 0.02-0.05% | Wetting Agent |
| Titanium Dioxide | 0.07-1.1% | 0.08-1.0% | Opacifier |
| Sodium Phosphate Monobasic | 0.4-0.9% | 0.5-0.8% | Buffer |
| Monohydrate | | | |
| Sodium Phosphate Dibasic | 0.8-1.3% | 0.9-1.2% | Buffer |
| Heptahydrate | | | |
| Flavoring agent | 0.2-0.6% | 0.3-0.5% | Flavor |
| Sucrose | 17.3-18.5% | 17.5-18.0% or | Sweetener |
| | | 17.5-18.5% | |
| Xylitol | 7.5-10% | 8.5-9.50% | Sweetener |
| Purified Water | q.s. | q.s. | Primary solvent |
| | Formulation B3 | Formulation B4 | |
| Components | Percentage (w/w) | Percentage (w/w) | Function |
| Fexofenadine zwitterionic | 0.3-0.7% | 0.4-0.6% | Active substance |
| hydrate Form I | | | |
| Propylene Glycol | 1.8-2.5% | 1.9-2.4% | Co-Solvent |
| Edetate Disodium dihydrate | 0.05-0.2% | 0.10-0.18% | Preservative Aid |
| Domiphen Bromide | 0.0001-10% | 0.001-1.0% | Preservative |
| Xanthan Gum | 0.1-0.5% | 0.2-0.4% | Suspending Agent |
| Poloxamer 407 | 0.01-0.07% | 0.02-0.05% | Wetting Agent |
| Titanium Dioxide | 0.07-1.1% | 0.08-1.0% | Opacifier |
| Sodium Phosphate Monobasic | 0.4-0.9% | 0.5-0.8% | Buffer |
| Monohydrate | | | |
| Sodium Phosphate Dibasic | 0.8-1.3% | 0.9-1.2% | Buffer |
| Heptahydrate | | | |
| Flavoring agent | 0.2-0.6% | 0.3-0.5% | Flavor |
| Sucrose | 17.3-18.5% | 17.5-18.0% or | Sweetener |
| | | 17.5-18.5% | |
| Xylitol | 7.5-10% | 8.5-9.50% | Sweetener |
| Purified Water | q.s. | q.s. | Primary solvent |

| | Formulation B5 | Formulation B6 | |
|---|---|---|---|
| Components | Percentage (w/w) | Percentage (w/w) | Function |
| Fexofenadine zwitterionic | 0.3-0.7% | 0.4-0.6% | Active substance |
| hydrate Form I | | | |
| Propylene Glycol | 1.8-2.5% | 1.9-2.4% | Co-Solvent |
| Edetate Disodium dihydrate | 0.05-0.2% | 0.10-0.18% | Preservative Aid |
| Cetylpyridium chloride | 0.0001-1% | 0.0005-0.01% | Preservative |
| Xanthan Gum | 0.1-0.5% | 0.2-0.4% | Suspending Agent |
| Poloxamer 407 | 0.01-0.07% | 0.02-0.05% | Wetting Agent |
| Titanium Dioxide | 0.07-1.1% | 0.08-1.0% | Opacifier |
| Sodium Phosphate Monobasic | 0.4-0.9% | 0.5-0.8% | Buffer |
| Monohydrate | | | |
| Sodium Phosphate Dibasic | 0.8-1.3% | 0.9-1.2% | Buffer |
| Heptahydrate | | | |
| Flavoring agent | 0.2-0.6% | 0.3-0.5% | Flavor |
| Sucrose | 17.3-18.5% | 17.5-18.0% or | Sweetener |
| | | 17.5-18.5% | |
| Xylitol | 7.5-10% | 8.5-9.50% | Sweetener |
| Purified Water | q.s. | q.s. | Primary solvent |

| | Formulation B7 | Formulation B8 | |
|---|---|---|---|
| Components | Percentage (w/w) | Percentage (w/w) | Function |
| Fexofenadine zwitterionic | 0.3-0.7% | 0.4-0.6% | Active substance |
| hydrate Form I | | | |
| Propylene Glycol | 1.8-2.5% | 1.9-2.4% | Co-Solvent |
| Edetate Disodium dihydrate | 0.05-0.2% | 0.10-0.18% | Preservative Aid |
| Sodium Benzoate | 0.0010%- | 0.03-0.8% | Preservative |
| | 10.00% | | |
| Xanthan Gum | 0.1-0.5% | 0.2-0.4% | Suspending Agent |
| Poloxamer 407 | 0.01-0.07% | 0.02-0.05% | Wetting Agent |
| Titanium Dioxide | 0.07-1.1% | 0.08-1.0% | Opacifier |
| Sodium Phosphate Monobasic | 0.4-0.9% | 0.5-0.8% | Buffer |
| Monohydrate | | | |
| Sodium Phosphate Dibasic | 0.8-1.3% | 0.9-1.2% | Buffer |
| Heptahydrate | | | |
| Flavoring agent | 0.2-0.6% | 0.3-0.5% | Flavor |
| Sucrose | 17.3-18.5% | 17.5-18.0% or | Sweetener |
| | | 17.5-18.5% | |
| Xylitol | 7.5-10% | 8.5-9.50% | Sweetener |
| Purified Water | q.s. | q.s. | Primary solvent |

In one embodiment, the method of preparing the paraben-free aqueous pharmaceutical suspension comprises
(a) adding pre-dissolved components of the sweetener system in purified water;
(b) adding a dispersion of the suspending agent in polypropylene to purified water,
(c) adding a portion of the buffer system to maintain pH control;
(d) adding the preservative system;
(e) dispersing the fexofenadine in an aqueous solution of the remaining components of the buffer system and the wetting agent;
(f) adding the dispersion of the fexofenadine;
(g) optionally adding an opacifying agent;
(h) adding the flavoring agent;
(i) adding purified water to adjust total volume;
(j) homogenizing the mixture; and
(k) milling and de-aerating the mixture.

### Embodiments

The invention provides also the following non-limiting embodiments.

Embodiment 1 is a paraben-free aqueous pharmaceutical suspension, having a pH 5.8 to 7.0, comprising:
0.03% to 1.20% (w/w) of fexofenadine zwitterionic dihydrate Form I of formula (I)

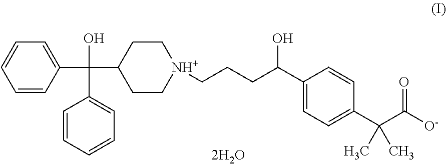 [SMILES: CC(C)(C(=O)[O-])c1ccc(C(O)CCC[NH+]2CCC(C(O)(c3ccccc3)c3ccccc3)CC2)cc1]

0.01% to 0.20% (w/w) of a wetting agent;
a suspending agent comprising 0.10% to 0.50% (w/w) of a hydrocolloid gum,
a sweetener system comprising 5% to 40% (w/w) of sucrose, and
5% to 40% (w/w) of xylitol, provided that the ratio of the amount of (sucrose) (xylitol) is 1:1 to 2:1;
a preservative system comprising (a) one of 0.0001% to 10.0% (w/w) domiphen bromide, 0.0001% to 10.0% (w/w) of cetylpyridinium chloride, 0.4% to 10% of sodium benzoate, 0.25% to 2% (w/w) of potassium sorbate, and (b) 0.01% to 0.25% (w/w) of edetate disodium;
a buffer system comprising 0.06% to 1.05% (w/w) of sodium phosphate monobasic or a corresponding equivalent amount of a sodium phosphate monobasic hydrate, or 0.06% to 1.05% (w/w) of potassium phosphate monobasic, and
0.32% to 2.69% (w/w) of sodium phosphate dibasic or a corresponding equivalent amount of a sodium phosphate dibasic hydrate, or 0.32% to 2.69% (w/w) of potassium phosphate dibasic or a corresponding equivalent amount of a potassium phosphate dibasic hydrate;
1.0% to 10.0% (w/w) of propylene glycol; and
0.20% to 0.70% (w/w) of a flavoring agent,
   wherein the aqueous pharmaceutical suspension is paraben-free.

Embodiment 2 is the paraben-free aqueous pharmaceutical suspension according to embodiment 1, wherein the preservative system comprises 0.0001% to 10.0% (w/w) domiphen bromide and 0.01% to 0.25% (w/w) of edetate disodium.

Embodiment 3 is the paraben-free aqueous pharmaceutical suspension according to embodiment 1, wherein the preservative system comprises 0.0001% to 10.0% (w/w) of cetylpyridinium chloride bromide and 0.01% to 0.25% (w/w) of edetate disodium.

Embodiment 4 is the paraben-free aqueous pharmaceutical suspension according to embodiment 1, wherein the preservative system comprises 0.4% to 10% of sodium benzoate and bromide and 0.01% to 0.25% (w/w) of edetate disodium.

Embodiment 5 is a paraben-free aqueous pharmaceutical suspension, having a pH 5.8 to 7.0, comprising:
0.03% to 1.20% (w/w) of fexofenadine zwitterionic dihydrate Form I of formula (I)

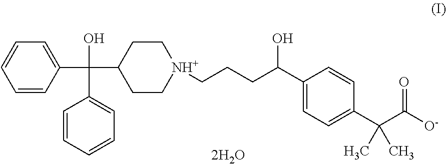 [SMILES: CC(C)(C(=O)[O-])c1ccc(C(O)CCC[NH+]2CCC(C(O)(c3ccccc3)c3ccccc3)CC2)cc1]

0.01% to 0.20% (w/w) of a wetting agent;
a suspending agent comprising 0.10% to 0.50% (w/w) of a hydrocolloid gum,
a sweetener system comprising 5% to 40% (w/w) of sucrose, and
5% to 40% (w/w) of xylitol, provided that the ratio of the amount of (sucrose) (xylitol) is 1:1 to 2:1;
a preservative system comprising 0.25% to 2% (w/w) of potassium sorbate, and
0.01% to 0.25% (w/w) of edetate disodium;
a buffer system comprising 0.06% to 1.05% (w/w) of sodium phosphate monobasic or a corresponding equivalent amount of a sodium phosphate monobasic hydrate, or 0.06% to 1.05% (w/w) of potassium phosphate monobasic, and
0.32% to 2.69% (w/w) of sodium phosphate dibasic or a corresponding equivalent amount of a sodium phosphate dibasic hydrate, or 0.32% to 2.69% (w/w) of potassium phosphate dibasic or a corresponding equivalent amount of a potassium phosphate dibasic hydrate;
1.0% to 10.0% (w/w) of propylene glycol; and
0.20% to 0.70% (w/w) of a flavoring agent,
wherein the aqueous pharmaceutical suspension is paraben-free.

Embodiment 6 is the paraben-free aqueous pharmaceutical suspension of any one of embodiments 1 to 4, wherein the suspension comprises 0.03% to 1.20% of fexofenadine zwitterionic dihydrate Form I of formula (I) having a particle size of less than 280 μm for at least 90% of the fexofenadine zwitterionic dihydrate Form I.

Embodiment 7 is the paraben-free aqueous pharmaceutical suspension according to embodiment 6, wherein the particle size is less than 50 μm for at least 90% of the fexofenadine zwitterionic dihydrate Form I.

Embodiment 8 is the paraben-free aqueous pharmaceutical suspension according to embodiment 7, wherein the particle size is less than 40 μm for at least 90% of the fexofenadine zwitterionic dihydrate Form I.

Embodiment 9 is the paraben-free aqueous pharmaceutical suspension according to any one of embodiments 1 to 8, wherein the fexofenadine zwitterionic dihydrate Form I is 0.3% to 0.7% (w/w).

Embodiment 10 is the paraben-free aqueous pharmaceutical suspension according to embodiment 9, wherein the fexofenadine zwitterionic dihydrate Form I is 0.4% to 0.6% (w/w).

Embodiment 11 is the paraben-free aqueous pharmaceutical suspension according to any one of embodiments 1 to 10, wherein the wetting agent is 0.01% to 0.07% (w/w).

Embodiment 12 is the paraben-free aqueous pharmaceutical suspension according to any one of embodiments 1 to 10, wherein the wetting agent is 0.02% to 0.05% (w/w).

Embodiment 13 is the paraben-free aqueous pharmaceutical suspension according to any one of embodiments 1 to 12, wherein the wetting agent is about 0.04% (w/w).

Embodiment 14 is the paraben-free aqueous pharmaceutical suspension according to any one of embodiments 1 to 12, wherein the wetting agent is ionic.

Embodiment 15 is the paraben-free aqueous pharmaceutical suspension according to embodiment 14, wherein the wetting agent is Poloxamer 188.

Embodiment 16 is the paraben-free aqueous pharmaceutical suspension according to embodiment 14, wherein the wetting agent is Poloxamer 407.

Embodiment 17 is the paraben-free aqueous pharmaceutical suspension according to any one of embodiments 1 to 16, wherein the hydrocolloid gum is 0.1% to 0.5% (w/w).

Embodiment 18 is the paraben-free aqueous pharmaceutical suspension according to any one of embodiments 1 to 16, wherein the hydrocolloid gum is, by 0.2% to 0.4% (w/w).

Embodiment 19 is the paraben-free aqueous pharmaceutical suspension according to any one of embodiments 1 to 18, wherein the hydrocolloid gum is about 0.31% (w/w).

Embodiment 20 is the paraben-free aqueous pharmaceutical suspension according to any one of embodiments 1 to 19, wherein the hydrocolloid gum is xanthan gum.

Embodiment 21 is the paraben-free aqueous pharmaceutical suspension according to any one of embodiments 1 to 20, wherein the xylitol is 7.5% to 10% (w/w).

Embodiment 22 is the paraben-free aqueous pharmaceutical suspension according to any one of embodiments 1 to 20, wherein the xylitol is 8.5% to 9.50% (w/w).

Embodiment 23 is the paraben-free aqueous pharmaceutical suspension according to any one of embodiments 1 to 22, wherein the sucrose is 17.3% to 18.5% (w/w).

Embodiment 24 is the paraben-free aqueous pharmaceutical suspension according to any one of embodiments 1 to 22, wherein the sucrose is 17.5% to 18.0% (w/w)

Embodiment 25 is the paraben-free aqueous pharmaceutical suspension according to any one of embodiments 1 to 24, wherein the potassium sorbate is 0.1% to 0.6% (w/w).

Embodiment 26 is the paraben-free aqueous pharmaceutical suspension according to embodiment 21, wherein the potassium sorbate is 0.15% to 0.5% (w/w).

Embodiment 27 is the paraben-free aqueous pharmaceutical suspension according to any one of embodiments 1 to 26, wherein the edetate disodium is 0.05% to 0.2% (w/w).

Embodiment 28 is the paraben-free aqueous pharmaceutical suspension according to any one of embodiments 1 to 26, wherein the edetate disodium is 0.10% to 0.18%.

Embodiment 29 is the paraben-free aqueous pharmaceutical suspension according to any one of embodiments 1 to 28, wherein the buffer system comprises 0.4% to 0.9% (w/w) of sodium phosphate monobasic or a corresponding equivalent amount of a sodium phosphate monobasic hydrate; and 0.5% to 0.8% (w/w) of sodium phosphate dibasic or a corresponding equivalent amount of a sodium phosphate dibasic hydrate.

Embodiment 30 is the paraben-free aqueous pharmaceutical suspension according to any one of embodiments 1 to 29, wherein the propylene glycol is 1.8% to 2.5% (w/w).

Embodiment 31 is the paraben-free aqueous pharmaceutical suspension according to any one of embodiments 1 to 29, wherein the propylene glycol is 1.9% to 2.4% (w/w).

Embodiment 32 is the paraben-free aqueous pharmaceutical suspension according to any one of embodiments 1 to 31, wherein the flavoring agent is 0.2% to 0.6% (w/w).

Embodiment 33 is the paraben-free aqueous pharmaceutical suspension according to any one of embodiments 1 to 31, wherein the flavoring agent is 0.3% to 0.5% (w/w).

Embodiment 34 is the paraben-free aqueous pharmaceutical suspension according to any one of embodiments 1 to 33, further comprising 0.07% to 1.1% (w/w) of an opacifying agent.

Embodiment 35 is the paraben-free aqueous pharmaceutical suspension according to any one of embodiments 1 to 33, further comprising 0.08% to 1.0% (w/w) of an opacifying agent.

Embodiment 36 is the paraben-free aqueous pharmaceutical suspension according to embodiment 34 or 35, wherein the opacifying agent is titanium dioxide.

Embodiment 37 is the paraben-free aqueous pharmaceutical suspension according to any one of embodiments 3 of 36, comprising 0.4% to 0.6% (w/w) of fexofenadine zwitterionic dihydrate Form I, 1.9% to 2.4% (w/w) of propylene glycol, 0.25% to 0.6% (w/w) potassium sorbate, 0.10% to 0.18% (w/w) of edetate disodium, 0.9% to 1.2% (w/w) of sodium phosphate dibasic heptahydrate, 0.5% to 0.8% (w/w) of sodium phosphate monobasic monohydrate, 0.2% to 0.4% (w/w) of xanthan gum, 0.02% to 0.05% (w/w) Poloxamer 407, 0.08% to 1.0% (w/w) of titanium dioxide, 17.5% to 18.0% (w/w) of sucrose, 8.5 to 9.50% (w/w) of xylitol, and 0.3% to 0.5% (w/w) of a flavoring agent.

Embodiment 38 is a paraben-free aqueous pharmaceutical suspension, having a pH 5.8 to 7.0, comprising: 0.4% to 0.6% (w/w) of fexofenadine zwitterionic dihydrate Form I of formula (I)

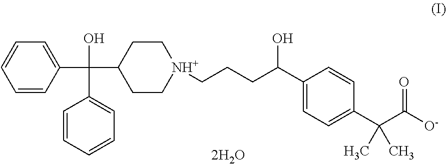 [SMILES: CC(C)(C(=O)[O-])c1ccc(C(O)CCC[NH+]2CCC(C(O)(c3ccccc3)c3ccccc3)CC2)cc1]

0.02% to 0.05% (w/w) of Poloxamer 407;
a suspending agent comprising 0.2% to 0.4% xanthan gum;
a sweetener system comprising 17.5% to 18.0% (w/w) of sucrose, and 8.5% to 9.50% (w/w) of xylitol;
a preservative system comprising 0.25% to 0.6% (w/w) potassium sorbate, and 0.10% to 0.18% (w/w) of edetate disodium;
a buffer system comprising 0.9% to 1.2% (w/w) of sodium phosphate dibasic heptahydrate, and 0.5% to 0.8% (w/w) of sodium phosphate monobasic monohydrate;
1.9% to 2.4% (w/w) of propylene glycol;
0.08% to 1.0% (w/w) of titanium dioxide; and
0.3% to 0.5% (w/w) of a flavoring agent,
wherein the aqueous pharmaceutical suspension is paraben-free.

Embodiment 39 is the paraben-free aqueous pharmaceutical suspension according to any one of the preceding embodiments, wherein the flavoring agent is artificial raspberry cream flavor or artificial orange cream flavor.

Embodiment 40 is a kit comprising a bottle or vial containing the paraben-free aqueous pharmaceutical suspension according to any one of embodiments 1 to 35 and a syringe or cup.

Embodiment 41 is use of the paraben-free aqueous pharmaceutical suspension according to any one of embodiments 1 to 39 for relieving symptoms due to an allergy such as an upper respiratory allergy in a patient in need thereof.

Embodiment 42 is the use according to embodiment 41, wherein the allergy is due to an indoor allergen such as dust mites, a pet allergen such as in animal saliva, animal urine or animal dander, or mold or outdoor allergen such as pollen such as from grass, weeds, or trees, such as hay fever, or mold.

Embodiment 43 is the paraben-free aqueous pharmaceutical suspension according to any one of embodiments 1 to 39 for relieving symptoms due to an allergy such as an upper respiratory allergy in a patient in need thereof.

Embodiment 44 is the paraben-free aqueous pharmaceutical suspension according to embodiment 43 wherein the allergy is due to an indoor allergen such as dust mites, a pet allergen such as in animal saliva, animal urine or animal dander, or mold or outdoor allergen such as pollen such as from grass, weeds, or trees, such as hay fever, or mold.

Embodiment 44 is a method of relieving symptoms due to an allergy in a patient in need thereof, comprising administering a therapeutically effective amount of the paraben-free aqueous pharmaceutical suspension according to any one of embodiments 1 to 39 to the patient.

Embodiment 46 is the method according to embodiment 45, wherein the patient is an adult.

Embodiment 47 is the method according to embodiment 46, wherein the patient is a child.

Embodiment 48 is the method according to any one of embodiments 45 to 47, wherein the allergy is due to an indoor allergen or outdoor allergen.

Embodiment 49 is the method according to claim 48, wherein the indoor allergen is dust mites, a pet allergen such as in animal saliva, animal urine or animal dander, or mold.

Embodiment 50 is the method according to claim 48, wherein the outdoor allergen is pollen such as from grass, weeds, or trees, such as hay fever, or mold.

Embodiment 51 is the method of any one of embodiments 45 to 50, wherein the symptom is one or more of red eye, itchy eye, watery eye, itchy nose, runny nose, stuffy nose, sneezing, nasal congestion, wheezing, coughing, chest tightness, facial pain, rash, hives, shortness of breath, cough, postnasal drip, itchy throat, dry skin, sinus pressure, decreased sense of smell, decreased sense of taste, or poor sleep quality.

Embodiment 52 is a method of preparing the paraben-free aqueous pharmaceutical suspension according to any one of embodiments 1 to 39, comprising
(a) adding pre-dissolved components of the sweetener system in purified water;
(b) adding a dispersion of the suspending agent in polypropylene to purified water,
(c) adding a portion of the buffer system to maintain pH control;
(d) adding the preservative system;
(e) dispersing the fexofenadine in an aqueous solution of the remaining components of the buffer system and the wetting agent;
(f) adding the dispersion of the fexofenadine;
(g) optionally adding an opacifying agent;
(h) the flavoring agent;
(i) adding purified water to adjust total volume;
(j) homogenizing the mixture; and
(k) milling and de-aerating the mixture

Without further description, it is believed that one of ordinary skill in the art can, using the preceding description and the following illustrative examples, make and utilize the present invention and practice the claimed methods. The following working examples, therefore, specifically point out the preferred embodiments of the present invention, and are not to be construed as limiting in any way the remainder of the disclosure.

### EXAMPLES

### Example 1—Generation of Fexofenadine Suspension Formulations

Sucrose and xylitol are added to purified water and dissolved in a jacketed main compounding tank. Xanthan gum in propylene glycol is added slowly and uniformly dispersed. With the recirculation loop on, the dispersion is transferred to a jacketed main compounding tank containing purified water and sweetener system that is previously heated to 35-45° C., and mixed. The batch is continually mixed through the end of processing. The temperature is maintained to the preservative addition step. The vessel is rinsed with the remaining propylene glycol and a portion of purified water, and the rinse is transferred to the main compounding tank. Portions of sodium phosphate dibasic heptahydrate and sodium phosphate monobasic monohydrate are added to the jacketed vessel and dissolved. The potassium sorbate and edetate disodium are added to the jacketed vessel and dissolved. The solution in the jacketed vessel is cooled to 20-30° C. The pH of the solution is measured.

In a separate vessel, the remaining portions of sodium phosphate dibasic heptahydrate and sodium phosphate monobasic monohydrate are added to purified water and dissolved with a high shear mixer. Poloxamer 407 is added and dissolved. The pH of the solution is measured. Anhydrous Form I of fexofenadine hydrochloride is added slowly to the solution and a uniform dispersion is formed. Titanium dioxide is added slowly to the dispersion and a uniform dispersion is formed. The dispersion is transferred to the solution in the jacketed main compounding tank. The tank containing the dispersion is rinsed with a portion of purified water and transferred to the main compounding tank. The raspberry cream flavor is added with a pressure can to the main compounding tank and dissolved. Sufficient purified water is added, if necessary to achieve the target net weight (10,000 kg).

Mixing is continued and a uniform suspension is formed. The pH is measured. The suspension is milled (i.e., URSCITEL® milled) and de-aerated. The resulting suspension contains 30 mg of Fexofenadine zwitterionic dihydrate Form I (converted from anhydrous Form I of fexofenadine hydrochloride) per 5 mL of suspension.

The components of the existing fexofenadine suspension formulations are shown in Table 1-1 below. In the existing formulation, EDTA is a chelator that has synergy with the preservative system to inhibit the effect of metals/ions and prevent the degradation of the components of the formula, ensuring the quality and preservation of the final product.

TABLE 1-1
Components of existing Fexofenadine suspension formulation

| Composition | Function |
|---|---|
| Fexofenadine HCl | API |
| Sodium Monobasic Phosphate | Buffer (pH 5.8-7.0) |
| Sodium Dibasic Phosphate | Buffer (pH 5.8-7.0) |
| Propyl Paraben | Preservative |
| Butyl Paraben | Preservative |
| Edetate Disodium | Synergistic effect with Parabens |
| Sucrose | Sweetener |
| Xylitol | Sweetener |
| Propylene Glycol | Co Solvent |
| Xanthan Gum | Suspending agent |
| Poloxamer 407 | Wetting agent |
| Raspberry Cream Flavor or vanilla | Flavoring agent |
| Titanium Dioxide | Opacifier |
| Purified Water | Solvent |

### Example 2—Generation of Paraben-Free Suspension Formulations

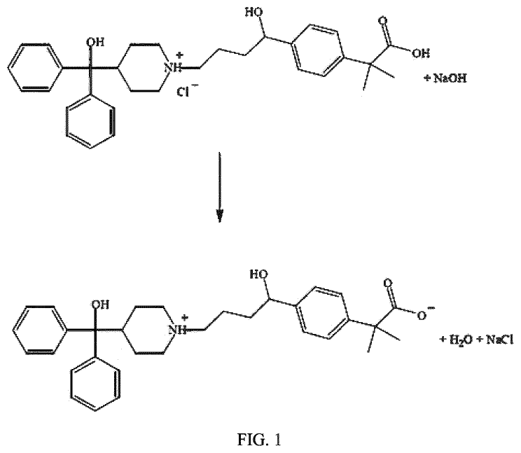

Fexofenadine and its pharmaceutically acceptable salts are useful as antihistamines as disclosed in U.S. Pat. No. 4,254,129, the contents of which is incorporated herein in its entirety. The Fexofenadine anhydrous form (I) is used in the manufacture of the Allegra® suspension because it is converted by means of a reaction to the base and Form I Zwitterion dihydrate (FIG. 1) which does not present in the product the bitter taste that is characteristic of the anhydrous form. The conversion of anhydrous form to the zwitterion shape is controlled by the pH of the suspension, which is monitored throughout the manufacture of the product, and controlled by the addition of monobasic sodium phosphate buffers and dibasic sodium phosphate. The ideal pH for maintaining the organoleptic characteristics of the product is between 5.8 and 7.0, whose 97% of fexofenadine is in the form of zwitterion.

The current preservative system of the Allegra® suspension consists of preservatives propylparaben and butylparaben, being a system commonly used for oral liquid pharmaceutical forms, having microbial activity and stability in the pH of the suspension (≈6.2), and broad spectrum of activity. The formulation and testing of the Allegra® suspension is disclosed in International Publication No. WO 2007/070517 and U.S. Pat. No. 8,933,097, the contents of which is incorporated herein in its entirety. The formulation is also shown in Table 1-1 above.

The existing formulation as disclosed in International Publication No. WO 2007/070517, U.S. Pat. No. 8,933,097, and Table 1-1 above contains parabens and sodium edetate (EDTA). This existing formulation is highly optimized. As shown in Table 1-1 above, edetate disodium is known to be synergistic with the parabens against certain microorganisms.

Parabens may be detrimental; thus, testing was conducted to identify a paraben-free preservative system with no impact on the quality and safety of the aqueous composition, maintaining the organoleptic characteristics of the suspension and the zwitterion form of Fexofenadine. In fact, use of butyl-paraben is currently banned in the European Union but not in other regions. The goal of the screening was to replace butyl-paraben with another preservative system and to avoid propyl-paraben while maintaining all the other characteristics of the formulation.

### Initial Screening

The selection of the new preservative system was initiated through bibliographic research and 55 preservatives approved for pharmaceutical products were identified. Of these, 29 preservatives were suitable for use in oral products, but most had pH efficacy below 5.0 outside of the preferred pH of the existing formulation.

Thus, the preservatives that were most promising, considering the target pH range of the product were: Methyl Paraben, Methyl Paraben Sodium, Methyl Paraben Potassium, Ethyl paraben, Ethyl paraben sodium, Potassium Ethyl Paraben, Butyl paraben, Sodium butyl paraben, Propyl paraben, Sodium propyl paraben, Potassium propyl paraben, Domiphen bromide, Propyl gallate, Sodium benzoate, Cetylpyridinium chloride, and Potassium sorbate.

### Further Testing

After eliminating paraben derivatives, according to the scope of the project, the following four preservatives were selected for further testing: Domiphen bromide, Sodium benzoate, Cetylpyridinium chloride, and Potassium sorbate.

### Example 3—Testing of Paraben Replacements

The four selected preservatives (Domiphen bromide, Sodium benzoate, Cetylpyridinium chloride, and Potassium sorbate) were tested in lab batches with more than 276 lab batches produced, more than 1,535 analytical tests conducted and approximately 143 preservative efficacy studies. These studies were used to identify suitable paraben replacements that exhibited similar preservative efficacy as the synergistic combination of preservatives in the existing formulation. To generate the formulations, the parabens of the existing formulations were replaced with the tested putative paraben replacement. The formulation stayed otherwise the same. As such, all of the preservatives were tested in combination with EDTA.

### Results

### Domiphen Bromide

Domiphen bromide showed good preservative system efficacy test (PET) results (see Table 3-1 and 3-2 below). Since domiphen bromide currently has no approval in the United States due to lack of toxicological studies no further studies were conducted.

TABLE 3-1
Results of the preservative efficacy test considering the criteria
adopted for the European pharmacopoeia (Ph. Eur).
Domiphen bromide

| Batch number | 104 | 105 | 106 |
|---|---|---|---|
| Preservative concentration | 0.0025% | 0.0050% | 0.0075% |
| Test conclusion | comply | comply | comply |

TABLE 3-2
Results of the preservative efficacy test considering the criteria
adopted for the US Pharmacopoeia (USP).
Domiphen bromide

| Batch number | 104 | 105 | 106 |
|---|---|---|---|
| Preservative concentration | 0.0025% | 0.0050% | 0.0075% |
| Test conclusion | comply | comply | comply |

### Cetylpyridinium Chloride

A pilot batch was manufactured in an industrial scale using cetylpyridinium chloride and stability studies were conducted up to 24 months, presenting satisfactory results (see Table 3-3 and Table 3-4). Since cetylpyridinium chloride currently does not have FDA approval for use in oral infant solutions no further studies were conducted.

TABLE 3-3
Results of the preservative efficacy test considering the
criteria adopted for the European pharmacopoeia (Ph. Eur).
Cetylpyridinium chloride

| Batch number | 152 | 153 | 154 | 183 | 184 | 185 | 186 |
|---|---|---|---|---|---|---|---|
| Preservative | 0.0075% | 0.0050% | 0.0025% | 0.0020% | 0.0015% | 0.0010% | 0.0005% |
| concentration | | | | | | | |
| Test conclusion | comply | comply | comply | comply | comply | comply | not |
| | | | | | | | comply |

### Sodium Benzoate

The studies conducted with this preservative system showed satisfactory results, but the change in the pH necessary to ensure its microbiological action altered the organoleptic characteristics (flavor and odor) of the product (see Table 3-5 and Table 3-6).

TABLE 3-5
Results of the preservative efficacy test considering the
criteria adopted for the European pharmacopoeia (Ph. Eur).

| | Sodium benzoate + NaOH (low pH) | |
|---|---|---|
| Batch number | 137 | 139 |
| Preservative concentration | 0.2% | 0.5% |
| Test conclusion | comply | comply |

TABLE 3-6
Results of the preservative efficacy test considering
the criteria adopted for the US Pharmacopoeia (USP).

| | Sodium benzoate + NaOH (low pH) | |
|---|---|---|
| Batch number | 137 | 139 |
| Preservative concentration | 0.2% | 0.5% |
| Test conclusion | comply | comply |

### Potassium Sorbate

Unexpectedly despite potassium sorbate being previously identified only as a preservative aid, potassium sorbate proved to be a suitable preservative. Initial studies showed that paraben-free suspensions using potassium sorbate preservative system presented yellowing during the accelerated stability study at 24 and 36 months. The investigation on the alteration of the color of the suspension was initiated with the comparison of the coloration of the paraben-free suspension.

### Example 4—Further Studies Using Potassium Sorbate as the Preservative

Further studies to test use of potassium sorbate for efficacy as an antimicrobial preservative were carried out in the formula with potassium sorbate in order to confirm the minimum concentration of use and definition of the specification, furthering the American and European monographs, in order to meet the different regulatory requirements of the countries in which this new formula will be submitted. The stability studies were conducted for a period of 36 months.

TABLE 4-1
The criteria adopted from both pharmacopoeias

| Class of microorganism | Ph. Eur - Section 5.1.3. | USP <51> |
|---|---|---|
| Bacteria | No less than 3 log10 of initial | Not less than 1.0 log10 |
| | count reduction in 14 days, | reduction of the initial count in |
| | and no increase in the count of | 14 days, and no increase in the |
| | 14 days in 28 days. | count of 14 days in 28 days. |
| Fungus and yeast | Not less than 1 log10 | No increase in initial |
| | reduction of the initial count in | calculated count at 14 and 28 |
| | 14 days, and no increase in the | days. |
| | count of 14 days in 28 days. | |

TABLE 4-2
Results of the preservative efficacy test considering the
criteria adopted for the European pharmacopoeia (Ph. Eur).

| Batch number | IN004-14/223 | IN004-14/224 | IN004-14/222 | IN004-14/217 | IN004-14/220 |
|---|---|---|---|---|---|
| Potassium Sorbate | 0.4% | 0.35% | 0.30% | 0.25% | 0% |
| Concentration | | | | | |
| Test Conclusion | Passed | Passed | Failed | Failed | Failed |

TABLE 4-3
Results of the preservative efficacy test considering the
criteria adopted for the American pharmacopoeia (USP).

| | Batch Number | | | | | | |
|---|---|---|---|---|---|---|---|
| | IN004- | IN004- | IN004- | IN004- | IN004- | IN004- | IN004- |
| | 14/220 | 14/223 | 14/224 | 14/222 | 14/217 | 14/218 | 14/219 |
| Potassium Sorbate | 0.4% | 0.35% | 0.30% | 0.25% | 0.20% | 0.10% | 0% |
| Concentration | | | | | | | |
| Test Conclusion | Approved | Approved | Approved | Approved | Approved | Not | Not |
| | | | | | | approved | approved |

Considering the results presented above, potassium sorbate was considered effective as a preservative at the concentration of 0.4%0 for considering the criteria of both pharmacopoeias, and for European pharmacopoeia the point of failure was with the concentration of 0.3% and for American pharmacopoeia 0.10, due to the difference in specifications between the tests.

Potassium sorbate showed to be effective until 36 months of stability.

Surprisingly and despite the known synergy between paraben and EDTA, it was found that a suspension formulation of fexofenadine using a preservative system containing only potassium sorbate and EDTA is bioequivalent to a solution containing parabens.

It was observed that using a preservative system containing only potassium sorbate and EDTA, it is possible to reduce the amount of total preservative in a suspension formulation of fexofenadine.

While the invention has been described and illustrated herein by references to various specific materials, procedures, and examples, it is understood that the invention is not restricted to the particular combinations of material and procedures selected for that purpose. Numerous variations of such details can be implied as will be appreciated by those skilled in the art. It is intended that the specification and examples be considered as exemplary, only, with the true scope and spirit of the invention being indicated by the following claims. All references, patents, and patent applications referred to in this application are herein incorporated by reference in their entirety.

1. A paraben-free aqueous pharmaceutical suspension, having a pH 5.8 to 7.0, comprising:
0.03% to 1.20% (w/w) of fexofenadine zwitterionic dihydrate Form I of formula (I) 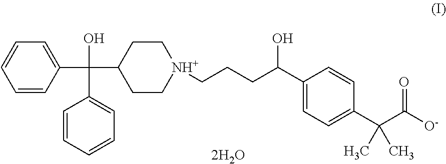 [SMILES: CC(C)(C(=O)[O-])c1ccc(C(O)CCC[NH+]2CCC(C(O)(c3ccccc3)c3ccccc3)CC2)cc1]
0.01% to 0.20% (w/w) of a wetting agent;
a suspending agent comprising 0.10% to 0.50% (w/w) of a hydrocolloid gum,
a sweetener system comprising 5% to 40% (w/w) of sucrose, and
5% to 40% (w/w) of xylitol, provided that the ratio of the amount of (sucrose):(xylitol) is 1:1 to 2:1;
a preservative system comprising 0.25% to 2% (w/w) of potassium sorbate, and
0.01% to 0.25% (w/w) of edetate disodium;
a buffer system comprising 0.06% to 1.05% (w/w) of sodium phosphate monobasic or a corresponding equivalent amount of a sodium phosphate monobasic hydrate, or 0.06% to 1.05% (w/w) of potassium phosphate monobasic, and
0.32% to 2.69% (w/w) of sodium phosphate dibasic or a corresponding equivalent amount of a sodium phosphate dibasic hydrate, or 0.32% to 2.69% (w/w) of potassium phosphate dibasic or a corresponding equivalent amount of a potassium phosphate dibasic hydrate;
1.0% to 10.0% (w/w) of propylene glycol; and
0.20% to 0.70% (w/w) of a flavoring agent,
wherein the aqueous pharmaceutical suspension is paraben-free.

2. A paraben-free aqueous pharmaceutical suspension, having a pH 5.8 to 7.0, comprising:
0.03% to 1.20% (w/w) of fexofenadine zwitterionic dihydrate Form I of formula (I) 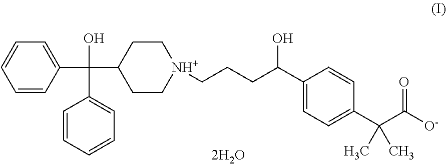 [SMILES: CC(C)(C(=O)[O-])c1ccc(C(O)CCC[NH+]2CCC(C(O)(c3ccccc3)c3ccccc3)CC2)cc1]
0.01% to 0.20% (w/w) of a wetting agent;
a suspending agent comprising 0.10% to 0.50% (w/w) of a hydrocolloid gum,
a sweetener system comprising 5% to 40% (w/w) of sucrose, and
5% to 40% (w/w) of xylitol, provided that the ratio of the amount of (sucrose):(xylitol) is 1:1 to 2:1;
a preservative system comprising (a) one of 0.0001% to 10.0% (w/w) domiphen bromide, 0.0001% to 10.0% (w/w) of cetylpyridinium chloride, 0.4% to 10% of sodium benzoate, 0.25% to 2% (w/w) of potassium sorbate, and (b) 0.01% to 0.25% (w/w) of edetate disodium;
a buffer system comprising 0.06% to 1.05% (w/w) of sodium phosphate monobasic or a corresponding equivalent amount of a sodium phosphate monobasic hydrate, or 0.06% to 1.05% (w/w) of potassium phosphate monobasic, and
0.32% to 2.69% (w/w) of sodium phosphate dibasic or a corresponding equivalent amount of a sodium phosphate dibasic hydrate, or 0.32% to 2.69% (w/w) of potassium phosphate dibasic or a corresponding equivalent amount of a potassium phosphate dibasic hydrate;
1.0% to 10.0% (w/w) of propylene glycol; and
0.20% to 0.70% (w/w) of a flavoring agent,
wherein the aqueous pharmaceutical suspension is paraben-free.

3. The paraben-free aqueous pharmaceutical suspension according to claim 2, wherein the preservative system comprises 0.0001% to 10.0% (w/w) domiphen bromide and 0.01% to 0.25% (w/w) of edetate disodium.

4. The paraben-free aqueous pharmaceutical suspension according to claim 2, wherein the preservative system comprises 0.0001% to 10.0% (w/w) of cetylpyridinium chloride bromide and 0.01% to 0.25% (w/w) of edetate disodium.

5. The paraben-free aqueous pharmaceutical suspension according to claim 2, wherein the preservative system comprises 0.4% to 10% of sodium benzoate and bromide and 0.01% to 0.25% (w/w) of edetate disodium.

6. The paraben-free aqueous pharmaceutical suspension according to claim 1, wherein the suspension comprises 0.03% to 1.20% of fexofenadine zwitterionic dihydrate Form I of formula (I) having a particle size of less than 280 m for at least 90% of the fexofenadine zwitterionic dihydrate Form I.

7. The paraben-free aqueous pharmaceutical suspension according to claim 6, wherein the particle size is less than 50 μm for at least 90% of the fexofenadine zwitterionic dihydrate Form I.

8. The paraben-free aqueous pharmaceutical suspension according to claim 7, wherein the particle size is less than 40 μm for at least 90% of the fexofenadine zwitterionic dihydrate Form I.

9. The paraben-free aqueous pharmaceutical suspension according claim 1, wherein the fexofenadine zwitterionic dihydrate Form I is 0.3% to 0.7% (w/w).

10. The paraben-free aqueous pharmaceutical suspension according to claim 9, wherein the fexofenadine zwitterionic dihydrate Form I is 0.4% to 0.6% (w/w).

11. The paraben-free aqueous pharmaceutical suspension according to claim 1, wherein the wetting agent is 0.02% to 0.05% (w/w).

12. The paraben-free aqueous pharmaceutical suspension according to claim 1, wherein the wetting agent is ionic.

13. The paraben-free aqueous pharmaceutical suspension according to claim 12, wherein the wetting agent is Poloxamer 188.

14. The paraben-free aqueous pharmaceutical suspension according to claim 12, wherein the wetting agent is Poloxamer 407.

15. The paraben-free aqueous pharmaceutical suspension according to claim 1, wherein the hydrocolloid gum is 0.2% to 0.4% (w/w).

16. The paraben-free aqueous pharmaceutical suspension according to claim 1, wherein the hydrocolloid gum is xanthan gum.

17. The paraben-free aqueous pharmaceutical suspension according to claim 1, wherein the xylitol is 7.5% to 10% (w/w).

18. The paraben-free aqueous pharmaceutical suspension according to claim 17, wherein the xylitol is 8.5% to 9.50% (w/w).

19. The paraben-free aqueous pharmaceutical suspension according to claim 1, wherein the sucrose is 17.3% to 18.5% (w/w).

20. The paraben-free aqueous pharmaceutical suspension according to claim 19, wherein the sucrose is 17.5% to 18.0% (w/w).

21. The paraben-free aqueous pharmaceutical suspension according to claim 1, wherein the potassium sorbate is 0.1% to 0.6% (w/w).

22. The paraben-free aqueous pharmaceutical suspension according to claim 21, wherein the potassium sorbate is 0.15% to 0.5% (w/w).

23. The paraben-free aqueous pharmaceutical suspension according to claim 1, wherein the edetate disodium is 0.05% to 0.2% (w/w).

24. The paraben-free aqueous pharmaceutical suspension according to claim 1, wherein the buffer system comprises 0.4% to 0.9% (w/w) of sodium phosphate monobasic or a corresponding equivalent amount of a sodium phosphate monobasic hydrate; and 0.5% to 0.8% (w/w) of sodium phosphate dibasic or a corresponding equivalent amount of a sodium phosphate dibasic hydrate.

25. The paraben-free aqueous pharmaceutical suspension according to claim 1, wherein the propylene glycol is 1.8% to 2.5% (w/w).

26. The paraben-free aqueous pharmaceutical suspension according to claim 1, wherein the flavoring agent is 0.2% to 0.6% (w/w).

27. The paraben-free aqueous pharmaceutical suspension according to claim 1, further comprises 0.07% to 1.1% (w/w) of an opacifying agent.

28. The paraben-free aqueous pharmaceutical suspension according to claim 27, wherein the opacifying agent is titanium dioxide.

29. The paraben-free aqueous pharmaceutical suspension according to claim 6, comprising 0.4% to 0.6% (w/w) of fexofenadine zwitterionic dihydrate Form I, 1.9% to 2.4% (w/w) of propylene glycol, 0.25% to 0.6% (w/w) potassium sorbate, 0.10% to 0.18% (w/w) of edetate disodium, 0.9% to 1.2% (w/w) of sodium phosphate dibasic heptahydrate, 0.5% to 0.8% (w/w) of sodium phosphate monobasic monohydrate, 0.2% to 0.4% (w/w) of xanthan gum, 0.02% to 0.05% (w/w) Poloxamer 407, 0.08% to 1.0% (w/w) of titanium dioxide, 17.5% to 18.0% (w/w) of sucrose, 8.5 to 9.50% (w/w) of xylitol, and 0.3% to 0.5% (w/w) of a flavoring agent.

30. The paraben-free aqueous pharmaceutical suspension according to claim 1, wherein the flavoring agent is artificial raspberry cream flavor or artificial orange cream flavor.

31. A kit comprising a bottle or vial containing the paraben-free aqueous pharmaceutical suspension according to claim 1 and a syringe or cup.

32-35. (canceled)

36. A method of relieving symptoms due to an allergy in a patient in need thereof, comprising administering a therapeutically effective amount of the paraben-free aqueous pharmaceutical suspension according to claim 1 to the patient.

37. The method according to claim 36, wherein the patient is an adult.

38. The method according to claim 37, wherein the patient is a child.

39. The method according to claim 36, wherein the allergy is due to an indoor allergen or outdoor allergen.

40. The method according to claim 39, wherein the indoor allergen is dust mites, a pet allergen such as in animal saliva, animal urine or animal dander, or mold.

41. The method according to claim 40, wherein the outdoor allergen is pollen such as from grass, weeds, or trees, such as hay fever, or mold.

42. The method of claim 36, wherein the symptom is one or more of red eye, itchy eye, watery eye, itchy nose, runny nose, stuffy nose, sneezing, nasal congestion, wheezing, coughing, chest tightness, facial pain, rash, hives, shortness of breath, cough, postnasal drip, itchy throat, dry skin, sinus pressure, decreased sense of smell, decreased sense of taste, or poor sleep quality.

43. A method of preparing the paraben-free aqueous pharmaceutical suspension according to claim 1, comprising
a) adding pre-dissolved components of the sweetener system in purified water;
b) adding a dispersion of the suspending agent in polypropylene to purified water,
c) adding a portion of the buffer system to maintain pH control;
d) adding the preservative system;
e) dispersing the fexofenadine in an aqueous solution of the remaining components of the buffer system and the wetting agent;
f) adding the dispersion of the fexofenadine;
g) optionally adding an opacifying agent;
h) adding the flavoring agent;
i) adding purified water to adjust total volume;
j) homogenizing the mixture; and
k) milling and de-aerating the mixture.
